(19) United States
(12) Patent Application Publication
(10) Pub. No.: US 2002/0115141 A1
(43) Pub. Date: Aug. 22, 2002

(54) Method for improved production of cyanophycin and secondary products thereof

(76) Inventors: Karl Ziegler, Berlin (DE); Wolfgang Lockau, Berlin (DE); Jan Ebert, Berlin (DE); Kirill Piotukh, Moskau (RU); Holger Berg, Berlin (DE); Rudolf Volkmer-Engert, Berlin (DE)

Correspondence Address: BAYER CORPORATION, PATENT DEPARTMENT, 100 BAYER ROAD, PITTSBURGH, PA, 15205, US

(21) Appl. No.: 09/923,563

(22) Filed: Aug. 7, 2001

(30) Foreign Application Priority Data
Aug. 9, 2000 (DE) 10038775.6
Jul. 27, 2001 (EP) PCT/EP01/08690

(51) Int. Cl. C12P 21/02; C12N 9/50; C12N 1/18; C12N 5/04; C12N 1/21

(52) U.S. Cl. 435/69.1; 435/219; 435/252.3; 435/254.2; 435/410

(57) ABSTRACT

The present invention relates to a thermostable cyanophycin synthetase produced from Synechococcus elongatus and to a method for improved production of cyanophycin and/or secondary products thereof.

### TECHNICAL FIELD OF THE INVENTION

The present invention relates to a thermostable cyanophycin synthetase, to transformed organisms containing such an enzyme and to a method for improved production of cyanophycin and/or secondary products thereof, for example polyaspartic acid or arginine.

### BACKGROUND OF THE INVENTION

Multi-L-arginyl-poly-L-aspartate (cyanophycin) is a branched polypeptide which contains aspartic acid and arginine in the ratio of 1:1. The chemical structure corresponds to a poly-α-aspartate backbone with arginine side radicals which are linked via peptide bonds to virtually all β-carboxyl groups of the backbone. DE-A 198 25 509 describes the identification, cloning and heterologous expression of the gene for cyanophycin synthetase from Synechocystis PCC 6803. The enzyme activity is determined here by means of a radioactive assay in which L-[U-¹⁴C]-arginine is incorporated into cyanophycin from Aphanocapsa PCC 6308, introduced as primer. The enzyme reaction itself takes place at 28° C. here.

DE-A 197 09 024 discloses the extraction and purification of cyanophycin from Aphanocapsa PCC 6308, the synthesis being carried out at 20° C.

DE-A 198 13 692 merely discloses isolation of the cyanophycin synthetase gene from Synechocystis PCC 6803 or Anabaena variabilis ATCC 29 413. Technical aspects of cyanophycin production, however, are not described here.

FEMS Microbiology Letters 181 (1999) 229-236 discloses the production of cyanophycin using Synechococcus sp. MA 19.

A disadvantage of large-scale cyanophycin production according to the known methods is that, for optimal product yield, a relatively narrow temperature range, normally below 35° C., should not be exceeded.

This represents a considerable restriction in the degrees of freedom for large-scale production within the process control for the production of cyanophycin, since higher temperatures from the outset prevent contamination by foreign cultures.

Therefore production of cyanophycin also at substantially higher temperatures than previously described, in combination with higher flexibility in process control and considerably improved product yields is desirable, in order to isolate therefrom the secondary products such as polyaspartic acid or arginine on a large scale.

This object is achieved by the present invention.

### SUMMARY OF THE INVENTION

The present invention relates to a cyanophycin synthetase which is distinguished by having a temperature optimum in the range of >35° C. and an amino acid sequence according to SEQ ID No: 01, encoded by an isolated nucleotide sequence according to SEQ ID No: 02, an allele, homologue or derivative of this nucleotide sequence or a nucleotide sequence hybridizing therewith.

In a preferred variant of the present invention, the cyanophycin synthetase of the invention has a temperature optimum in the range from 35° C. bis 55° C., preferably in the range from 35° C. bis 50° C.

The cyanophycin synthetase is further distinguished by the fact that it originates from Synechococcus elongatus. The cyanophycin synthetase of the invention represents a thermostable enzyme.

### BRIEF DESCRIPTION OF THE DRAWINGS

FIG. 1 is a representation of the chemical structure of the synthetic peptide primers used for synthesis of cyanophycin by means of cyanophycin synthetase.

FIG. 2 is a representation of the results of an SDS polyacrylamide gel electrophoresis (SDS-PAGE) for in vitro synthesis of cyanophycin-like material by means of purified cyanophycin synthetase.

FIG. 3 is a representation of the results of an SDS-PAGE for chain elongation of a primer by means of cyanophycin synthetase at the C-terminal end of the peptide primer.

### DETAILED DESCRIPTION OF THE PREFERRED EMBODIMENTS OF THE INVENTION

The present invention relates to a cyanophycin synthetase which is distinguished by having a temperature optimum in the range of >35° C. and an amino acid sequence according to SEQ ID No: 01, encoded by an isolated nucleotide sequence according to SEQ ID No: 02, an allele, homologue or derivative of this nucleotide sequence or a nucleotide sequence hybridizing therewith.

In a preferred variant of the present invention, the cyanophycin synthetase of the invention has a temperature optimum in the range from 35° C. bis 55° C., preferably in the range from 35° C. bis 50° C.

The cyanophycin synthetase is further distinguished by the fact that it originates from Synechococcus elongatus. The cyanophycin synthetase of the invention represents a thermostable enzyme.

The present invention also relates to isoenzymes of the cyanophycin synthetase of the invention. These isoenzymes mean enzymes having identical or comparable substrate specificity and action specificity, but having a different primary structure. In addition, the present invention also includes modified forms of cyanophycin synthetase. According to the invention, these mean enzymes in which alterations are present in the sequence, for example at the N and/or C termini of the polypeptide or in the region of conserved amino acids, which alterations, however, do not impair the function of the enzymes. These modifications may be carried out by exchanging one or more amino acids according to known methods.

A particular embodiment of the present invention includes variants of the inventive cyanophycin synthetase, whose substrate specificity, for example, was altered, for example with regard to the production of polyaspartic acid, by the amino acid exchange, compared with the particular starting protein. The same is true for the stability of the enzymes of the invention in cells; for example, the enzymes have increased or reduced sensitivity towards degradation by proteases.

The present invention further relates to polypeptides with cyanophycin synthetase function, whose amino acid sequence has been altered such that they are insensitive to regulatory compounds, for example to the metabolic endproducts regulating their activity (feedback insensitive).

An isolated nucleotide sequence or an isolated nucleic acid fragment means, according to the invention, an RNA or DNA polymer which may be single- or double-stranded and may optionally contain natural, chemically synthesized, modified or artificial nucleotides. The term “DNA polymer” here also includes genomic DNA, cDNA or mixtures thereof.

According to the invention, alleles mean functionally equivalent nucleotide sequences, i.e. nucleotide sequences with essentially identical action. Functionally equivalent sequences are those sequences which, despite deviating nucleotide sequences, for example due to the degeneracy of the genetic code, still retain the desired functions. Functional equivalents thus include naturally occurring variants of the sequences described herein and also to artificial nucleotide sequences obtained, for example, by chemical synthesis and, where appropriate, adjusted to the codon usage of the host organism. Moreover, functionally equivalent sequences include those having a modified nucleotide sequence which confers on the enzyme insensitivity or resistance to inhibitors, for example.

A functional equivalent means in particular also natural or artificial mutations of an originally isolated sequence which continue to show the desired function. Mutations include substitutions, additions, deletions, exchanges or insertions of one or more nucleotide residues. Also included here are “sense mutations” which can lead at the protein level to the exchange of conserved amino acids, for example, but not to any fundamental change in the protein activity and thus are functionally neutral. This also includes modifications of the nucleotide sequence which, at the protein level, concern the N or C terminus of a protein but with no substantial restriction of protein function. These modifications may even have a stabilizing influence on the protein structure.

The present invention further also includes those nucleotide sequences which are obtained by modification of the nucleotide sequence, resulting in corresponding derivatives. The aim of such a modification may be, for example, the further narrowing down of the coding sequence contained therein or else, for example, the introduction of further recognition sites for restriction enzymes. Functional equivalents are also those variants whose function, compared with the starting gene or gene fragment, is reduced or enhanced.

In addition, the present invention relates to artificial DNA sequences, as long as they provide the desired properties, as described above, and can be inserted into or appended to the gene of the cyanophycin synthetase of the invention. It is possible, for example, to determine such artificial DNA sequences by translating back from proteins generated by means of computer-assisted programs (molecular modelling) or by in vitro selection. Coding DNA sequences which have been obtained by translation back from a polypeptide sequence according to the codon usage specific for the host organism are particularly suitable. It is possible for a skilled worker familiar with molecular genetic methods readily to determine the specific codon usage by computer analyses of other, already known genes of the organism to be transformed.

According to the invention, homologous sequences mean those which are complementary to the nucleotide sequences of the invention and/or hybridize with these sequences. The term “hybridizing sequences” includes, according to the invention, substantially similar nucleotide sequences from the group comprising DNA or RNA, which specifically interact with (bind to) the abovementioned nucleotide sequences under known stringent conditions. This also includes short nucleotide sequences of, for example, from 10 to 30, preferably from 12 to 15 nucleotides in length. According to the invention, “nucleotide primers” or probes are inter alia also included here.

According to the invention, the sequence regions preceding (5′ or upstream) and/or following (3′ or downstream) the coding regions (structural genes) are also included. In particular, sequence regions with regulatory function are included here. They can influence transcription, RNA stability or RNA processing and also translation. Examples of regulatory sequences are inter alia promoters, enhancers, operators, terminators or translation enhancers.

Operative linkage means the sequential arrangement of, for example, promoter, coding sequence, terminator and, where appropriate, further regulatory elements, such that each of the regulatory elements can fulfil its predetermined function when the coding sequence is expressed. These regulatory nucleotide sequences may be of natural origin or can be obtained by chemical synthesis. A suitable promoter is in principle any promoter which is able to control gene expression in the appropriate host organism. According to the invention, the said promoter may also be a chemically inducible promoter which makes it possible to control at a particular time expression of the genes subject to it in the host cell. By way of example, mention may be made here of a promoter inducible by IPTG (isopropyl β-thiogalactopyranoside).

A gene construct is prepared by fusion of a suitable promoter with the nucleotide sequence of the invention, according to common recombination and cloning techniques known from the literature. The DNA fragments can be linked to one another by attaching adapters or linkers to the fragments.

Moreover, the present invention relates to a vector comprising at least one nucleotide sequence of the type described above coding for a cyanophycin synthetase specific for producing cyanophycin, regulatory nucleotide sequences operatively linked to the said nucleotide sequence and also additional nucleotide sequences for selection of transformed host cells, for replication within the host cell or for integration into the appropriate host cell genome. The vector of the invention may further comprise a gene construct of the abovementioned type.

Suitable vectors are those which are replicated in micro-organisms such as, for example, bacteria, fungi and/or plants. Examples of known vectors are pBluescript (Stratagene, 11099 North Torney Pines Rd., La Jolla, Calif. 92 037, USA) or pET (Novagen, 601 Science Drive, Madison, WJ 53 711, USA). This list, however, is non-limiting for the present invention.

Utilizing the nucleic acid sequences of the invention, it is possible to synthesize and use appropriate probes or else nucleotide primers for the purpose of amplifying and isolating analogous genes from other unicellular or multicellular organisms, preferably bacteria, fungi, algae or plants, for example with the aid of the polymerase chain reaction (“PCR”) technique.

The present invention thus also relates to a probe for identifying and/or isolating genes coding for proteins involved in cyanophycin biosynthesis, preferably further thermostable cyanophycin synthetases; this probe is prepared starting from the inventive nucleic acid sequences of the type described above and contains a label suitable for detection. The probe may be a section of the sequence of the invention, for example from a conserved region, which is, for example, from 10 to 30 or, preferably, 12 to 15 nucleotides in length and which can hybridize specifically with homologous nucleotide sequences under stringent conditions. Suitable labels are known from the literature in large numbers.

The present invention further relates to the transfer of the inventive nucleic acid sequence or a part thereof, coding for a cyanophycin synthetase, an allele, homologue or derivative thereof, or of a nucleotide sequence hybridizing with these sequences into a heterologous host system. This also includes the transfer of a gene construct or vector of the invention into a heterologous host system.

According to the invention, a heterologous host system means a unicellular or multicellular organism. Examples of these are micro-organisms, fungi, lower or higher plants, tissue or cells thereof. According to the invention, preference is given to bacteria, particularly preferably of the genus of enterobacteria and, in particular, of the species Escherichia coli. Furthermore, useful plants such as potatoes or tobacco are particularly preferred.

The inventive nucleotide sequence coding for an inventive thermostable cyanophycin synthetase is transferred into one of the abovementioned host systems according to known methods. Examples of methods for DNA transfer into suitable host systems, which may be mentioned, are transformation, electroporation, conjugation and agrobacteria-mediated DNA transfer or particle bombardment. This list serves only the purpose of illustrating the present invention and is non-limiting.

A transformed unicellular or multicellular organism resulting from a successful nucleic acid transfer thus differs from the corresponding untransformed organism by containing and being able to express additional nucleic acids of the inventive type.

The invention thus also relates to a transformed unicellular or multicellular organism comprising a cyanophycin synthetase of the invention and/or a vector comprising a cyanophycin synthetase of the type described above.

The present invention further relates to a method for providing an inventive cyanophycin synthetase of the type described above, in which method the nucleotide sequence coding for the enzyme is isolated from a thermophilic unicellular or multicellular organism, is, where appropriate, operatively linked to regulatory structures and/or cloned into a vector suitable for heterologous expression, is, where appropriate, transferred into a heterologous host system, is expressed there and is finally isolated from this host system and, where appropriate, purified and/or concentrated.

Direct isolation of an amount of cyanophycin synthetase which is sufficient for cyanophycin synthesis, from a thermophilic organism, without prior concentration in a heterologous system, is also conceivable. Furthermore, it is then possible to use the inventive cyanophycin synthetase enzyme, for example in an in vitro system for synthesizing cyanophycin and/or secondary products thereof.

The present invention also relates to a method for producing cyanophycin and/or secondary products thereof, in which a cyanophycin synthetase and/or a vector and/or a transformed unicellular or multicellular organism of the type described above are used. However, the present invention includes not only the production of cyanophycin and/or secondary products thereof in a living host system but also the in-vitro synthesis of cyanophycin with the aid of an isolated cyanophycin synthetase of the type described above.

The inventive method for producing cyanophycin is distinguished by carrying out the enzyme-catalyzed synthesis in a temperature range from 35° C. to 55° C., preferably in a range from 35° C. to 50° C.

The method of the invention is advantageously distinguished by the fact that, owing to the wide temperature range, the process is less error-prone, in particular above 28° C., allows greater variability in process control and thus provides improved product yield. Thus, the inventive production of cyanophycin and/or secondary products thereof is substantially more reproducible and economical than the hitherto known methods.

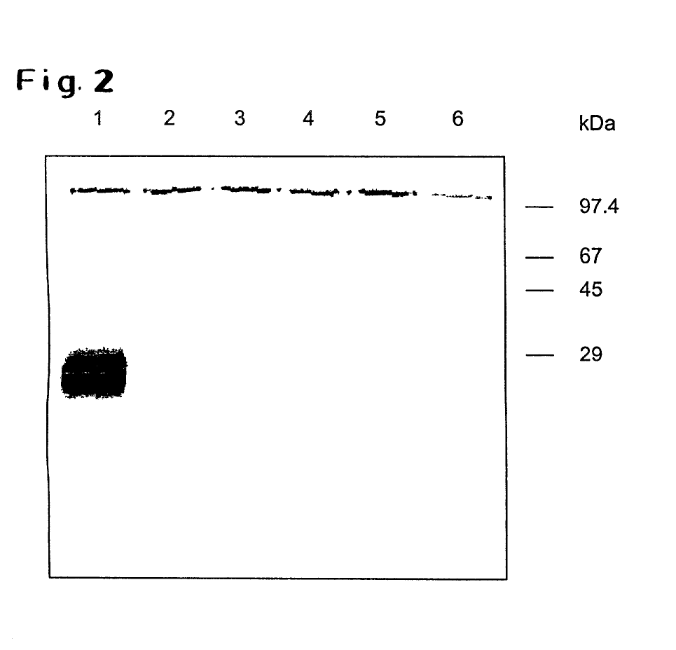

At the molecular level, the cyanophycin synthetase of the invention catalyses an ATP-dependent chain elongation. Surprisingly, the enzyme has two active (catalytic) centres. The cyanophycin synthetase of the invention stepwise and alternately (sequentially) incorporates one aspartic acid molecule and subsequently one arginine molecule into a cyanophycin precursor (peptide primer). Without a primer, the enzyme-catalysed chain elongation cannot be started. Studies thereon are depicted in FIG. 2.

Referring now to FIG. 2 there is illustrated a representation of the results of an SDS polyacrylamide gel electrophoresis (SDS-PAGE) for in vitro synthesis of cyanophycin-like material by means of purified cyanophycin synthetase. The reaction mixture contains inter alia about 10 μM Primer (β-Asp-Arg)₃. After incubation for 24 hours at room temperature, aliquots of the reaction mixture are analysed by means of SDS-PAGE and proteins are visualized according to standard methods. The lanes illustrate the following: Lane 1: complete reaction mixture; lane 2: reaction mixture without aspartic acid; lane 3: reaction mixture without arginine; lane 4: reaction mixture without ATP; lane 5: reaction mixture without primer (β-Asp-Arg)₃; lane 6: reaction mixture with heat-inactivated enzyme (5 min, 100° C.). The protein band above the 97.4 kDa standard represents cyanophycin synthetase. The diffuse bands below 29 kDa in lanes 1, 2 and 3 represent cyanophycin-like material.

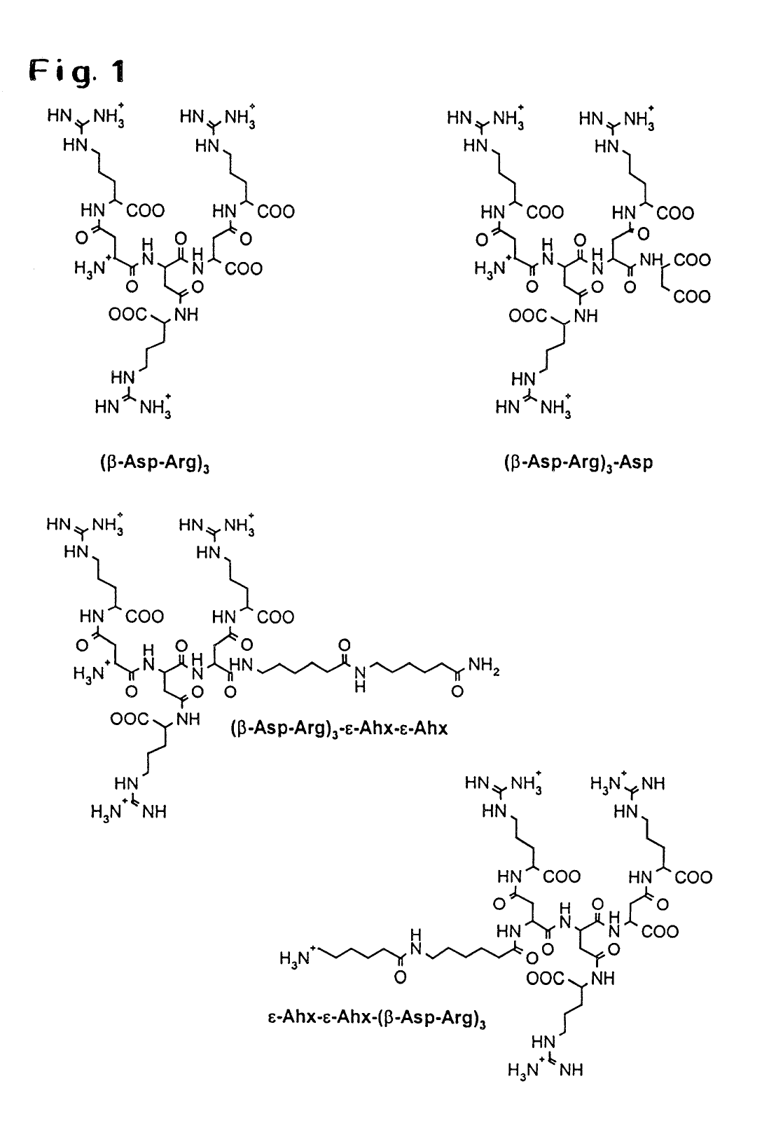

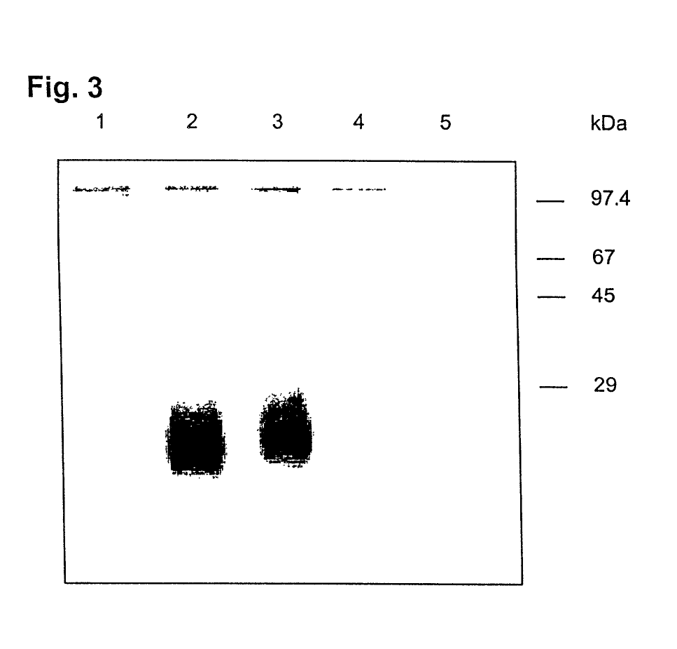

The chemical structure of various primers used in the synthesis of cyanophycin is depicted in FIG. 1. This clearly indicates that incorporation takes place exclusively at the C-terminal end of the precursor and only if both amino acids, i.e. aspartic acid and arginine or another basic amino acid, are present together. A summary of these studies is depicted in FIG. 3 and Table 1.

Referring now to FIG. 3 there is illustrated a representation of the results of an SDS-PAGE for chain elongation of a primer by means of cyanophycin synthetase at the C-terminal end of the peptide primer. Various primers (FIG. 1) are added to the reaction mixture. After incubation of the reaction mixtures for 24 hours at room temperature, aliquots of the reaction mixture are analysed by means of SDS-PAGE and proteins are visualized according to standard methods. The lanes illustrate the following: lane 1: mixture without primer; lane 2: mixture with about 8 μM unprotected primer (β-Asp-Arg)₃; lane 3: mixture with about 8 μM N-terminally protected primer (ε-Ahx₂-(β-Asp-Arg)₃); lane 4: mixture with about 8 μM C-terminally protected primer ((β-Asp-Arg)₃-ε-Ahx₂); lane 5: as lane 4 but with about 160 μM primer. The diffuse bands below 29 kDa in lanes 1, 2 and 3 represent cyanophycin-like material. Table 1 below illustrates the cyanophycin synthetase-catalysed incorporation of L-aspartic acid (Asp) and L-arginine (Arg) into synthetic peptide primers.

TABLE 1

| | | | |
|---|---|---|---|
| No. | Primer | Substrate(s) | Product |

C-terminally blocked primer:

| | | | |
|---|---|---|---|
| (1) | (β-Asp-Arg)₃-ε-Ahx₂ | Asp + Arg | none |
| (2) | (β-Asp-Arg)₃-ε-Ahx₂ | Asp or Arg | none |

N-terminally blocked primer:

| | | | |
|---|---|---|---|
| (3) | ε-Ahx₂-(β-Asp-Arg)₃ | Asp + Arg | Cyanophycin |
| (4) | ε-Ahx₂-(β-Asp-Arg)₃ | Asp | ε-Ahx₂-(β-Asp-Arg)₃-Asp |
| (5) | ε-Ahx₂-(β-Asp-Arg)₃ | Arg | none |

Unblocked primers:

| | | | |
|---|---|---|---|
| (6) | (β-Asp-Arg)₃ | Asp + Arg | Cyanophycin |
| (7) | (β-Asp-Arg)₃ | Asp | (β-Asp-Arg)₃-Asp^{a)} |
| (8) | (β-Asp-Arg)₃ | Arg | none |
| (9) | (β-Asp-Arg)₃ | β-Asp-Arg | none |
| (10) | (β-Asp-Arg)₃-Asp | Asp + Arg | Cyanophycin |
| (11) | (β-Asp-Arg)₃-Asp | Asp | none |
| (12) | (β-Asp-Arg)₃-Asp | Arg | (β-Asp-Arg)₄ |

^{a)}In principle, the reaction product could also be Asp-(β-Asp-Arg)₃. However, this possibility is excluded due to the results using the N-terminally or C-terminally blocked primers (reactions 2 and 4 of this table).

The present invention further relates to the use of a vector comprising an inventive cyanophycin synthetase of the abovementioned type for preparing a transformed unicellular or multicellular organism as described above. The present invention likewise includes the use of such a transformed unicellular or multicellular organism for producing an inventive cyanophycin synthetase and/or for producing cyanophycin and/or secondary products thereof. Moreover it is also possible to make use of a cyanophycin synthetase isolated according to the invention for in-vitro production of cyanophycin and/or secondary products thereof. In addition, the present invention relates to the use of cyanophycin and/or secondary products thereof for producing food supplements and/or compositions in the fields of agriculture and/or crop protection. Further fields of application for cyanophycin and/or secondary products thereof can be found in the paper, textile, pigment, paint, ceramics, building material or detergent industry and also in the fields of water and wastewater treatment.

The present invention is characterized in more detail by the following examples which are, however, not limiting for the invention:

General Genetic Methods:

DNA isolation, plaque hybridization, polymerase chain reaction (PCR), construction of a genomic DNA gene library and the procedures for protein analysis by means of SDS polyacrylamide gel electrophoresis (SDS-PAGE) including protein purification, as well as culturing of microorganisms such as, for example, Escherichia coli are carried out according to standard methods described in Sambrook, J. et al. (1998, Molecular Cloning: A Laboratory Manual; 2^{nd} Edition, Cold Spring Harbor Laboratory Press, N.Y.) or according to information by the manufacturers. Blue-green algae such as, for example, Synechococcus elongatus were cultured according to descriptions in Yamaoka, T., et al. (1978, Plant Cell Physiol., 19: 943-954).

Peptide Primer Synthesis:

The branched peptide primers (β-Asp-Arg)₃, (β-Asp-Arg)₃-Asp, ε-Ahx₂-(b-Asp-Arg)₃ and (β-Asp-Arg)₃-ε-Ahx₂ (see FIG. 1; Ahx=ε-aminohexane acid) were synthesized on a solid phase following Fmoc/tBu chemistry via O-(benzotriazol-1-yl)-N,N,N′,N′-tetramethyluronium tetrafluoroborate activation using the building block Fmoc-Asp-[Arg(Pmc)-OtBu]-OH. The building block was prepared in solution by the following reaction sequence: (i) acylation of H-Arg(Pmc)-OtBu (Bachem Biochemicals) with Fmoc-Asp-All (All=allyl ester) (Novabiochem) using dicyclohexylcarbodiimide/N-hydroxybenzotriazole (Novabiochem) as activators and then (ii) opening of the allyl ester with the aid of N-methylaniline and tetrakis(triphenylphosphine)palladium(0) as catalyst. The synthesis is started on a resin loaded with Fmoc-Arg(Pmc)-TentaGel-S-PHB (Rapp Polymere). The peptide primers are linked by the following reaction: (i) coupling of Fmoc-Asp-OtBu and subsequently (ii) attaching twice the building block Fmoc-Asp-[Arg-(Pmc)-OtBu]-OH. Furthermore, the N-terminally blocked peptide primer ε-Ahx₂-(b-Asp-Arg)₃ was prepared by attaching Fmoc-ε-aminohexane acid (Novabiochem) twice to the resin-bound peptide primer described above. The finished peptides were deprotected by treatment with 94% trifluoroacetic acid, 1% phenol, 2% water, 3% triisobutylsilane and removed from the resin, the peptides and the N-terminally blocked peptide primers being obtained as free acids. The C-terminally blocked peptide primer (β-Asp-Arg)₃-ε-Ahx₂ was prepared on a TentaGel-SRAM resin (Rapp-Polymere) according to the following procedure: (i) two times coupling of Fmoc-ε-aminohexane acid and subsequently three times coupling of the building block Fmoc-L-Asp-[L-Arg(Pmc)-OtBu). The finished primer was removed as described above and gave the peptide as carboxamide.

The peptide primer (β-Asp-Arg)₃-Asp was synthesized on a TentaGel-S-PHB resin (Rapp Polymere) loaded with Fmoc-Asp(OtBu) by attaching the appropriate building block three times. As described above the peptide was likewise removed from the resin and deprotected. The peptide was obtained as free acid here. All peptide primers were purified on a C-18 column (Vydac 201SP54) and analysed with the aid of RP HPLC and MALDI MS.

The dipeptide β-Asp-Arg was likewise prepared on a TentaGel-S-PHB phase which had been loaded with Fmoc-Arg(Pmc) before. After the Fmoc protection group had been removed with 20% strength piperidine-DMF solution, the resin was treated with 4 eq (equivalents) of Boc-Asp-OtBu (Bachem Chemicals), 4 eq of O-(benzotriazol-1-yl)-N,N,N′,N′-tetramethyluronium tetrafluoroborate and 8 eq of diisopropyl-ethylamine in DMF. The peptide was removed from the resin with trifluoroacetic acid containing 1% phenol, 2% water and 3% triisobutylsilane, then precipitated with cold t-butylmethyl and finally purified on a C-18 column (Vydac 201SP54) and analysed by RP HPLC and MALDI MS.

Reaction Mixtures and Product Analysis:

The reaction mixtures for product analysis by means of mass spectrometry contain in a volume of 125 μl the following components: 100 mM NH₄HCO₃ (pH 8.0), 4 mM ATP disodium salt, 20 mM MgCl₂, 8 mM KCl, 2 mM DTT, 0.2 mM L-aspartic acid, 0.2 mM L-arginine, ≧10 μM synthetic primers and 3 μg of cyanophycin synthetase.

For product analysis by means of SDS-PAGE, 125 μl of reaction mixture contain the following: 50 mM Tris-HCl (pH 8.0), 4 mM ATP disodium salt, 20 mM MgCl₂, 20 mM KCl, 1 mM DTT, 0.8 mM L-aspartic acid, 0.4 mM L-arginine, ≧10 μM synthetic primers and 3 μg of cyanophycin synthetase.

The samples are incubated at room temperature for 10-14 hours and subsequently either mixed with sample buffer (SDS-PAGE) or frozen (mass spectrometry). The products are analysed by means of mass spectrometry (MALDI MS) according to the information in the user manual of the manufacturer (PerSeptive Biosystems).

Other than in the operating examples, or where otherwise indicated, all numbers expressing quantities of ingredients, reaction conditions, etc. used in the specification and claims are to be under stood as modified in all instance by the term “about.”

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

We claim:

1. A cyanophycin synthetase comprising an amino acid sequence according to SEQ ID No: 01, encoded by a nucleotide sequence according to SEQ ID No: 02, an allele, homologue or derivative of said nucleotide sequence or a nucleotide sequence hybridizing therewith, and originating from Synechococcus elongatus,
wherein said cyanophycin synthetase has a temperature optimum of at least 35° C.

2. A vector comprising at least one nucleotide sequence coding for a cyanophycin synthetase according to claim 1 or 19, which is specific for cyanophycin production.

3. A transformed unicellular or multicellular organism comprising a cyanophycin synthetase according to claim 19 and/or a vector according to claim 2.

4. A transformed unicellular or multicellular organism according to claim 3, wherein said organism is selected from the group consisting of a microorganism, a fungus, a lower or higher plant, tissue or at least one cell therefrom.

5. A method for providing a cyanophycin synthetase according to claim 1 or 19, comprising the steps of operatively linking said nucleotide sequence coding for said cyanophycin synthetase to regulatory structures and/or is cloning said nucleotide sequence into a vector suitable for heterologous expression, transferring said nucleotide sequence into a heterologous host system and expressing and isolating and/or purifying and/or concentrating said cyanophycin synthetase from said host system.

6. A method for producing cyanophycin and the secondary products to be produced therefrom, comprising the step of employing a cyanophycin synthetase according to claim 1 or 19 and/or a vector according to claim 2 and/or a transformed unicellular or multicellular organism according to any of claims 3 and 4 to produce said cyanophycin.

7. A method for preparing a transformed unicellular or multicellular organism according to either of claims 3 and 4 comprising the step of employing a vector according to claim 2 to produce said transformed organism.

8. A method for producing a compound selected from the group consisting of a cyanophycin synthetase according to claim 1 or 19 and cyanophycin comprising the steps of providing the transformed unicellular or multicellular organism according to any of claims 3 to 4, whereupon said organism produces said cyanophycin synthetase and/or said cyanophycin.

9. A method for producing cyanophycin comprising the steps of providing said cyanophycin synthetase according to claim 1 or 19, and employing said cyanophycin synthetase to produce said cyanophycin.

10. Isoenzymes and modified forms of cyanophycin synthetase wherein said isoenzymes and said
modified forms of cyanophycin synthetase are obtained by modifying cyanophycin synthetasefrom Synechococcus elongatus and wherein said iso enzymes and modified forms of cyanophycin synthetase have a temperature optimum in the range of 35° C. to 55° C.

11. The isoenzymes and the modified forms of cyanophycin synthetase according to claim 10, wherein said isoenzymes and said modified forms of cyanophycin synthetase are obtained by amino acid exchange.

12. The isoenzymes and the modified forms of cyanophycin synthetase according to claim 11, wherein the amino acid exchange is carried out by modifying a nucleotide sequence of an underlying gene of said Synechococcus elongatus.

13. An artificial DNA sequence, comprising an artificial DNA sequence that is insertable into or appendable to a gene wherein said artificial DNA sequence encodes for and expresses the cyanophycin synthetase according to claim 1 or 19.

14. A probe for the identification and/or isolation of one or more genes coding for proteins involved in cyanophycin biosynthesis, said probe comprising a label suitable for detecting cyanophycin synthetase and modifications thereof according to claims 1 and 10.

15. A heterologous host system comprising a nucleic acid sequence or a part thereof coding for a member of the group consisting of cyanophycin synthetase, an isoenzyme and modified forms thereof according to claims 1, 19 or 10.

16. A method for the synthesis of polyaspartic acid or arginine comprising the steps of providing said cyanophycin synthetase according to claim 1 or 19 and employing said cyanophycin synthetase to produce said polyaspartic acid or said arginine.

17. Cyanophycin synthetase comprising a DNA sequence selected from the group consisting of a natural DNA sequence and an artificial DNA sequence, said DNA sequence being located between the 5′ or upstream and/or 3′ or downstream position of the cyanophycin synthetase according to claim 1, wherein said DNA sequences influence transcription, RNA stability of RNA processing, and translation.

18. The synthetase of claim 1 wherein said synthetase has a temperature optimum in the range of about 35° C. to about 55° C.

19. The synthetase of claim 1 wherein said synthetase has a temperature optimum in the range of 35° C. to 55° C.

20. The synthetase of claim 1 wherein said synthetase has a temperature optimum in the range of 35° C. to 50° C.

21. A polypeptide having cyanophycin synthetase functionality comprising a cyanophycin synthetase having a temperature optimum of at least 35° C. and wherein at least one amino acid sequence of said polypeptide has been altered such that said polypeptide is rendered insensitive to the regulating action of regulatory compounds that would otherwise regulate the activity of said polypeptide with respect to said polypeptide's cyanophycin synthetase functionality.

22. The polypeptide of claim 21 wherein said cyanophycin synthetase is a cyanophycin synthetase according to claims 1 or 19.


SEQUENCE LISTING (2 sequences)

SEQ ID NO: 1 - protein, 896 aa, Synechococcus elongatus
MKILKLQTLRGPNYWSIRRHKLIVMRLDLEEVANTPSNQISGFVDGLVRVLPSLYNHFCSLGHEGGFLTRLREGTYLGHVVEHVALELQELAGMPVGFGRTRETSTPGVYQVVYEYQVEEAGRYAGRAAVRLCQSIIDTGTYPQQELDQDLADLRELKAKASLGPSTEAIVREAEARNIPWFELSSRSIIQLGYGARSHRMQATLSDRSSILAVELASDKEGAKRLLQDAGIPVPKGTVIRYIEDLPEAIEEIGGYPIVIKPLNGNHGRGITIDINSLEAAEEAFEIASSISKSVIVERYHAGRDFRVLVVNGKVVAVAERVPAHVIGDGHSTIEELIEKTNQDPQRGDGHDNILTRIEVNHDTWTLLEKQGYTLNTVLQPGEICYLRATANLSTGGIAIDRTDEIHPENVWICQRAARIIGLDIAGIDVVSPDISQPLSKVGGVIVEVNAAPGFRMHTNPSQGIARNVAEPVLNMLFPPGTPCRIPIFAITGTNGKTTTTRLIAHICKQTGQTVGYTTTDGIYIGDYLVEKGDTTGPQSAQLILQDPTVEIAVLETARGGILRSGLGFDHCDVGVVLNVQADHLGLGDIDTVEQLADLKAVVVESAWPNGYAVLNADDPLVAAMARQVKAQVAYFSMDPHNPIIRQHIQQGGLAAVYENGYLSILKGDWTLRIEQAENVPITLGARASFMIANALAASLAAFAQGISIEHIRAALTTFRTSVEQTPGRMNLFDLGQFSVLVDYAHNPAGYEAIGEFVQKWPGQRIGVVGGPGDRRDQDLEQLGELSAKIFDWIIIKEDDDTRGRPRGDAAYWIERGVHHHSVQRQYDIIHDEVAAIQFALDRAPKGSLVVIFPAEVSRTIQLIRQHHQRLQGETINGFHSEGRPTSGDLNPSIFH

SEQ ID NO: 2 - DNA, 2691 bp, Synechococcus elongatus
  CDS (1)..(2691): cphA (cyanophycin synthetase)
atgaagattctcaaattacaaacgctgcggggtcccaattactggagcattcggcgtcataagctgattgtcatgcgtttagatctagaagaggtggccaacaccccctccaatcagatttctgggtttgtggatgggttggtgcgggttttgccgagtctttacaatcatttttgttctctcggacacgaagggggctttctcacccgcctccgagaaggtacgtatcttggtcatgtggttgaacatgttgccctcgagctccaagaactggcagggatgcccgttggctttggccgcacgcgggagacctcaacgccgggggtgtatcaagtggtctatgaataccaagtggaagaagcgggccgctatgccggccgagcagcagtgcgactgtgccaaagtattattgatacgggtacctatccccagcaagaactggatcaggatctcgccgatctccgggagttgaaagcaaaagcctcccttggcccgagtacggaagcgattgtccgcgaagccgaagcccgcaacatcccttggtttgagttgagcagtcgctcgattattcaattgggctatggcgcccgcagtcatcggatgcaagccacattgagcgatcgcagtagcatcttggcagttgaactcgccagtgacaaagaaggggcaaagcgactgcttcaggatgcgggaattcccgtgcctaagggaaccgtcatccgctatattgaagacctccccgaggccattgaggagatcggtggctatcccattgtcattaagcccctcaacggcaaccacggtcgcgggattacgattgacatcaacagcctagaagcagccgaagaagcctttgaaattgccagcagcatctccaaatccgtcattgtggaacgctatcatgccggtcgcgacttccgcgttctagtggtcaatggcaaagtggttgctgttgctgaacgggtgccggcccatgtgattggcgatggccactccaccatcgaagaactcattgagaaaacgaaccaagacccgcaacggggagacggtcacgataatatcctcacccgcattgaagtcaaccacgacacttggacactcctggaaaaacagggctataccctgaatacggtcttgcaaccgggggaaatttgttatctacgggccacggcgaacctaagtactggtggcattgccatcgatcgcactgatgaaattcacccggaaaatgtttggatttgccagcgggctgctcggatcattggcctcgatattgctggtattgacgttgtcagccccgatattagtcagcccctgtctaaagttggcggtgtgattgtcgaggtcaatgccgctcctggctttcgcatgcacaccaaccccagccaagggattgcccgcaatgttgccgaaccggtgttgaatatgctctttccaccgggaacaccttgccgcatcccgatctttgccattacggggaccaatggcaaaaccaccaccacccgtctcattgcccatatctgcaaacaaacggggcaaaccgttggctacaccaccacagacggcatctatattggcgattatctggtggaaaaaggagacaccaccggcccccaaagtgcccaactgatcctgcaggaccccaccgttgagatcgccgttctcgaaacggcgcgaggtgggattctccgctccggcttgggctttgaccattgtgatgtcggggtggtgctcaatgtgcaggctgatcaccttggccttggcgatattgacaccgttgagcagttggcggacttaaaggcagtggtggtggaatctgcttggccaaatggctacgctgtgttgaatgccgatgatcccctagtggcggcaatggcacgccaagtcaaagctcaagtggcctatttctcgatggatccccacaatcccatcattcggcagcacatccagcaggggggactcgccgctgtttatgaaaatggctacctctcaattttgaaaggggactggacactgcgcattgagcaggcagaaaatgtgcccattacccttggcgctcgagcaagctttatgattgccaatgccctcgctgccagtctagcggcctttgcccaaggcatcagtattgagcatattcgcgccgccttgaccaccttccgaacctcggtggagcaaacccccggtcggatgaacctctttgatttggggcaatttagtgtcttggtggactatgcccacaatccagcagggtatgaagccattggtgaatttgtccagaaatggccagggcagcgcattggtgtcgttggcggaccaggcgatcgccgcgatcaagacttggagcaactgggggaactctcggcgaaaatttttgattggatcatcattaaggaagatgatgatacccgtggccggcctcggggcgatgccgcctattggattgagcggggggtacatcaccacagtgtccagcggcaatacgacatcatccatgacgaggtggcagcgattcaatttgccctcgatcgcgctcccaaaggatccttagtggtgatctttccagcggaagtcagccgcacgattcaactgattcgccagcatcaccaacgactccaaggggaaacgatcaatggctttcacagtgagggaaggcccaccagtggtgatctcaacccctccatctttcattag